(19) United States
(12) Patent Application Publication
(10) Pub. No.: US 2023/0076824 A1
(43) Pub. Date: Mar. 9, 2023

(54) INFORMATION PROCESSING DEVICE AND INFORMATION PROCESSING METHOD

(71) Applicant: CANON MEDICAL SYSTEMS CORPORATION, Otawara-shi (JP)

(72) Inventors: Yasuko FUJISAWA, Nasushiobara (JP); Takuya SAKAGUCHI, Utsunomiya (JP)

(73) Assignee: CANON MEDICAL SYSTEMS CORPORATION, Otawara-shi (JP)

(21) Appl. No.: 17/929,476

(22) Filed: Sep. 2, 2022

(30) Foreign Application Priority Data
Sep. 6, 2021 (JP) 2021-144995

(51) Int. Cl. G16H 10/20; G16H 10/60

(52) U.S. Cl. CPC G16H 10/20; G16H 10/60

(57) ABSTRACT

An information processing device of an embodiment includes a processing circuitry. The processing circuitry estimates a future living situation of a target patient for each treatment method to be applied to the target patient on the basis of attributes of the target patient. The processing circuitry outputs information based on the living situation via an output unit.

### CROSS-REFERENCE TO RELATED APPLICATION

The present application claims priority based on Japanese Patent Application No. 2021-144995 filed on Sep. 6, 2021, the contents of which is incorporated herein by reference.

### FIELD

Embodiments disclosed in the present specification and drawings relate to an information processing device and an information processing method.

### BACKGROUND

Conventionally, a medical worker such as a doctor unilaterally decided a treatment direction for patients. In recent medical practice, understanding the intentions and values of a patient and his or her family, and a medical worker and a patient and his or her family determining a treatment direction together through an agreement has been conceived. Reaching an agreement with the patient and his or her family and then making a treatment direction is called Shared Decision Making.

There are the following issues in selecting a treatment method based on an intention of a patient or his or her family. In order for the patient and his or her family to envision their daily life after discharge from medical information, the patient and his or her family may lack information or knowledge or the medical information may not be able to be converted into information related to daily life in the first place, and there are thus information gaps. Because the selection of the treatment method itself comes first and does not take into account subsequent daily life, the selection can sometimes be a selection that it is difficult for the patient and his or her family to cope with. Because it may not be possible to clarify the priorities and the extent of toleration of the patient and his or her family, it is difficult to reach an agreement in consideration of envisioning future treatment.

In the related art, the life after treatment desired by the patient and his or her family is not input to the medical side, and an agreement regarding a treatment method, that is, the life after treatment through interaction with the medical side is lacking. Further, when forming an agreement between a patient and his or her family and the medical side, it may be difficult for the patient and his or her family to imagine an actual life from medical information, the patient and his or her family may not be able to make decisions, and the medical side may not notice information gaps.

### BRIEF DESCRIPTION OF THE DRAWINGS

FIG. 1 is a diagram illustrating a configuration example of an information processing system in a first embodiment.

FIG. 2 is a diagram illustrating a configuration example of a terminal device in the first embodiment.

FIG. 3 is a diagram illustrating an example of an intention input screen.

FIG. 4 is a diagram illustrating a configuration example of the information processing device in the first embodiment.

FIG. 5 is a flowchart illustrating a flow of a series of process of a processing circuitry according to the first embodiment.

FIG. 6 is a flowchart illustrating a flow of a process of estimating prognosis of a target patient.

FIG. 7 is a diagram illustrating an example of attribute distribution.

FIG. 8 is a diagram illustrating a stratification process.

FIG. 9 is a diagram illustrating a method of comparing attribute distributions.

FIG. 10 is a diagram illustrating an example of matters that are estimated as a future living situation.

FIG. 11 is a flowchart illustrating a flow of a series of process of a processing circuitry according to a second embodiment.

FIG. 12 is a diagram illustrating an example of a desired living situation.

FIG. 13 is a diagram illustrating an example of an estimated living situation.

FIG. 14 is a diagram illustrating an example in which an information gap has been generated.

FIG. 15 is a diagram illustrating an output example of a solution to the information gap.

### DETAILED DESCRIPTION

Hereinafter, an information processing device and an information processing method of an embodiment will be described with reference to the drawings.

The information processing device of the embodiment includes a processing circuitry. The processing circuitry estimate a future living situation of a target patient for each treatment method to be applied to the target patient on the basis of attributes of the target patient. The processing circuitry outputs information based on the living situation via an output unit. This makes it possible to select a treatment method suitable for an intention of a patient or his or her family.

### First Embodiment

### Configuration of Information Processing System

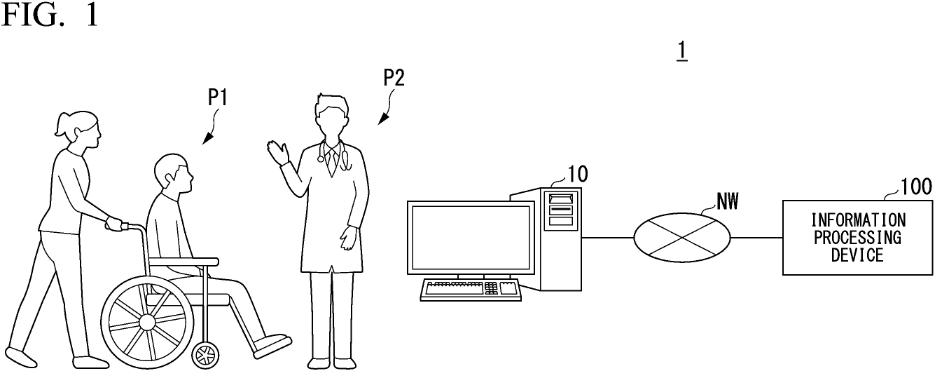

FIG. 1 is a diagram illustrating a configuration example of an information processing system 1 according to a first embodiment. The information processing system 1 includes, for example, a terminal device 10 and an information processing device 100. The terminal device 10 and the information processing device 100 are communicatively connected via a communication network NW.

The communication network NW may mean an entire information communication network using telecommunications technology. For example, the communication network NW includes a wireless/wired local area network (LAN) such as a hospital backbone LAN, an Internet network, a telephone communication line network, an optical fiber communication network, a cable communication network, a satellite communication network, and the like.

The terminal device 10 is a terminal device such as a personal computer, a tablet terminal, or a mobile phone that is used by a medical worker P2. The medical worker P2 is typically a doctor, but may be a nurse, another person involved in medical care, or a person involved in a community nursing service. The medical worker P2, for example, inputs information on a patient who is a treatment target (hereinafter referred to as a target patient P1) to the terminal device 10. Further, the target patient P1 or his or her family may input the information on the target patient P1 to the terminal device 10 instead of the medical worker P2 inputting the information on the target patient P1. Further, a family is target persons who input information on the family as with the patient.

The terminal device 10 transmits the information input by the medical worker P2 to the information processing device 100 or receives information from the information processing device 100 via the communication network NW.

The information processing device 100 receives information from the terminal device 10 via the communication network NW, and processes the received information. The information processing device 100 transmits the processed information to the terminal device 10 via the communication network NW.

The information processing device 100 may be a single device or may be a system in which a plurality of devices connected via the communication network NW operate in cooperation with each other. That is, the information processing device 100 may be realized by a plurality of computers (processors) included in a distributed computing system or a cloud computing system. Further, the information processing device 100 does not necessarily have to be a separate device from the terminal device 10, and may be a device integrated with the terminal device 10.

### Configuration of Terminal Device

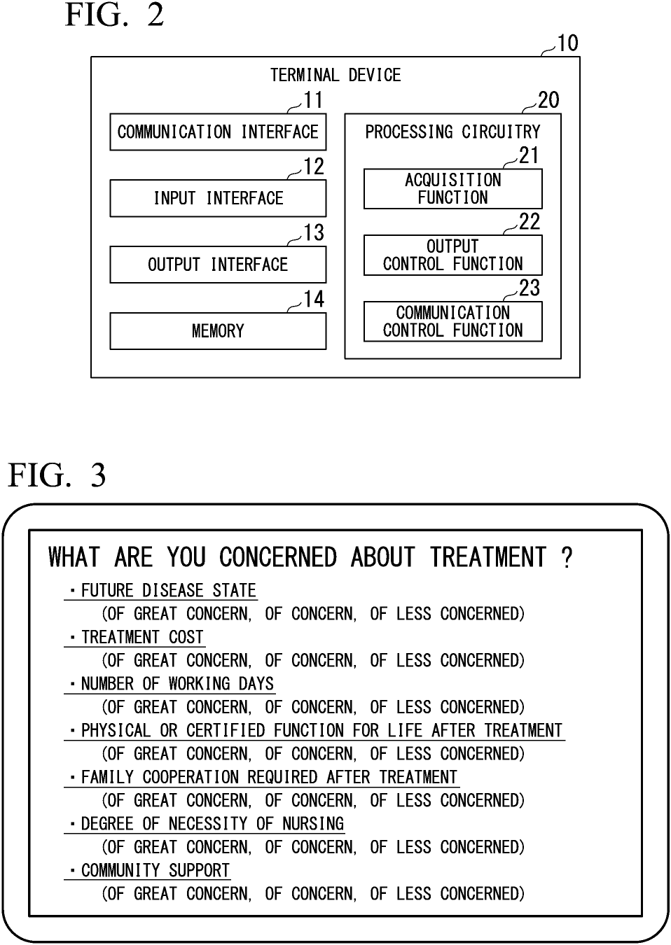

FIG. 2 is a diagram illustrating a configuration example of the terminal device 10 in the first embodiment. The terminal device 10 includes, for example, a communication interface 11, an input interface 12, an output interface 13, a memory 14, and a processing circuitry 20.

The communication interface 11 communicates with the information processing device 100 or the like via the communication network NW. The communication interface 11 includes, for example, a network interface card (NIC) or an antenna for wireless communication.

The input interface 12 receives various input operations from an operator (for example, the medical worker P2), converts the received input operations into electrical signals, and outputs the electrical signals to the processing circuitry 20. For example, the input interface 12 includes a mouse, a keyboard, a trackball, a switch, a button, a joystick, a touch panel, and the like. The input interface 12 may be, for example, a user interface that receives a voice input from a microphone or the like. When the input interface 12 is a touch panel, the input interface 12 may also have a display function of a display 13a to be described below.

In the present specification, the input interface 12 is not limited to a device including physical operation parts such as a mouse and a keyboard. For example, an example of the input interface 12 includes an electrical signal processing circuitry that receives an electrical signal corresponding to an input operation from an external input device provided separately from the device and outputs the electrical signal to a control circuit.

The output interface 13 outputs information under the control of the processing circuitry 20. For example, the output interface 13 includes the display 13a, a speaker 13b, and the like.

The display 13a displays various types of information. For example, the display 13a displays an image generated by the processing circuitry 20, a graphical user interface (GUI) for receiving various input operations from the medical worker P2, and the like. For example, the display 13a is a liquid crystal display (LCD), a cathode ray tube (CRT) display, an organic electro luminescence (EL) display, or the like.

The speaker 13b converts various types of information into sound and outputs the sound. For example, the speaker 13b outputs information input from the processing circuitry 20 as sound.

The memory 14 is realized by, for example, a random access memory (RAM), a semiconductor memory element such as a flash memory, a hard disk, or an optical disc. These non-transient storage media may be realized by other storage devices connected via the communication network NW, such as a network attached storage (NAS) and an external storage server device. Further, the memory 14 may include a non-transient storage medium such as a read only memory (ROM) or a register.

The processing circuitry 20 includes, for example, an acquisition function 21, an output control function 22, and a communication control function 23. The processing circuitry 20 realizes these functions by, for example, a hardware processor (a computer) executing a program stored in the memory 14 (a storage circuit).

The hardware processor in the processing circuitry 20 is, for example, a circuitry such as a central processing unit (CPU), a graphics processing unit (GPU), an application specific integrated circuit (ASIC), a programmable logic device (for example, a simple programmable logic device (SPLD), a complex programmable logic device (CPLD), or a field programmable gate array (FPGA)). The program may be directly embedded in a circuit of the hardware processor, instead of being stored in the memory 14. In this case, the hardware processor realizes functions by reading and executing the program embedded in the circuit. The program may be stored in the memory 14 in advance or may be stored in a non-transient storage medium such as a DVD or a CD-ROM and installed in the memory 14 from the non-transient storage medium by the non-transient storage medium being mounted in a drive device (not illustrated) of the information processing device 10. The hardware processor is not limited to one configured as a single circuit, and may be configured as one hardware processor that is a combination of a plurality of independent circuits to realize each function. Further, a plurality of components may be integrated into one hardware processor to realize each function.

The acquisition function 21 acquires input information via the input interface 12, or acquires information from the information processing device 100 via the communication interface 11.

The output control function 22 displays the information acquired by the acquisition function 21 as an image on the display 13a, or outputs the information as vocal sound from the speaker 13b.

For example, the output control function 22 causes a screen on which an intention of the target patient P1 or his or her family regarding treatment can be input (hereinafter referred to as an intention input screen) to be displayed on the display 13a. The intention input screen will be described below.

The communication control function 23 transmits the information input to the input interface 12 to the information processing device 100 via the communication interface 11.

FIG. 3 is a diagram illustrating an example of the intention input screen. Matters that may be of concern to the target patient P1 herself or himself or the family of the target patient P1 regarding a living situation (daily life) of the target patient P1 after the treatment can be input to the intention input screen. For example, on the intention input screen, questions regarding matters such as an economic and social environment surrounding the target patient P1 or his or her family or a medical expenses burden are asked in a questionnaire format or a free entry format. Specific pieces of content of the questions are, for example, (1) future disease state, (2) treatment cost, (3) number of working days, (4) physical or certified functions for life after treatment, (5) family cooperation required after treatment, (6) degree of necessity of nursing care, and (7) community support. For each question, scores can be input for degree of interest, acceptability and possibility of cooperation. For example, an option “of great concern” may indicate a highest degree of interest /a lowest degree of acceptability, an option “of concern” may indicate that the degree of interest is lower/the degree of acceptability is higher than in the option “of great concern”, and an option “of less concerned” indicates that the degree of interest is lower/the degree of acceptability is higher than the option “of concern”. Scores such as the degree of interest, the degree of acceptability, and the degree of cooperation are determined according to which option the target patient P1 or his or her family has answered among the three options. The number of questions is not limited to seven, and the options for each question are not limited to three.

### Configuration of Information Processing Device

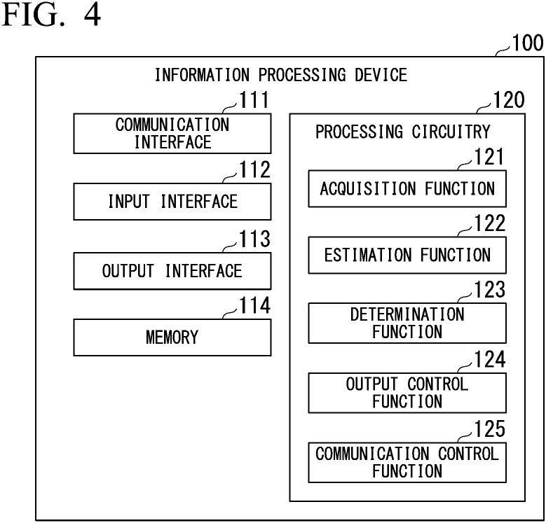

FIG. 4 is a diagram illustrating a configuration example of the information processing device 100 according to the first embodiment. The information processing device 100 includes, for example, a communication interface 111, an input interface 112, an output interface 113, a memory 114, and a processing circuitry 120.

The communication interface 111 communicates with the terminal device 10 or the like via the communication network NW. The communication interface 111 includes, for example, a NIC. The communication interface 111 is an example of an “output unit”.

The input interface 112 receives various input operations from the operator, converts the received input operations into electrical signals, and outputs the electrical signals to the processing circuitry 120. For example, the input interface 112 includes a mouse, a keyboard, a trackball, a switch, a button, a joystick, a touch panel, and the like. The input interface 112 may be, for example, a user interface that receives a voice input from a microphone or the like. When the input interface 112 is a touch panel, the input interface 112 may also have a display function of a display 113a that will be described below.

In the present specification, the input interface 112 is not limited to a device including physical operation parts such as a mouse and a keyboard. For example, an example of the input interface 112 includes an electrical signal processing circuitry that receives an electrical signal corresponding to an input operation from an external input device provided separately from the device and outputs the electrical signal to the control circuit.

The output interface 113 outputs information under the control of the processing circuitry 120. For example, the output interface 113 includes the display 113a, a speaker 113b, and the like. The output interface 113 is an example of an “output unit”.

The display 113a displays various types of information. For example, the display 13a displays an image generated by the processing circuitry 120, a GUI for receiving various input operations from the operator, and the like. For example, the display 113a is an LCD, a CRT display, an organic EL display, or the like.

The speaker 113b converts various types of information into sound and outputs the sound. For example, the speaker 113b outputs the information input from the processing circuitry 120 as sound.

The memory 114 is realized by, for example, a semiconductor memory element such as a RAM or a flash memory, a hard disk, or an optical disc. These non-transient storage media may be realized by other storage devices connected via the communication network NW such as a NAS or an external storage server device. Further, the memory 114 may include a non-transient storage medium such as a ROM or a register.

The processing circuitry 120 includes, for example, an acquisition function 121, an estimation function 122, a determination function 123, an output control function 124, and a communication control function 125. The estimation function 122 is an example of an “estimation unit”, and the determination function 123 is an example of a “determination unit”. When the output interface 113 is an example of the “output unit”, the output control function 124 is an example of an “output control unit”. Further, when the communication interface 111 is another example of the “output unit”, the communication control function 125 is another example of the “output control unit”.

The processing circuitry 120 realizes these functions, for example, by a hardware processor (a computer) executing a program stored in the memory 114 (a storage circuit).

The hardware processor in the processing circuitry 120 means, for example, a circuitry such as a CPU, a GPU, an integrated circuit for a specific application, a programmable logic device (for example, a simple programmable logic device or a complex programmable logic device), or a field programmable gate array. The program may be directly embedded in a circuit of the hardware processor, instead of being stored in the memory 114. In this case, the hardware processor realizes functions by reading and executing the program embedded in the circuit. The program may be stored in the memory 114 in advance or may be stored in a non-transient storage medium such as a DVD or a CD-ROM and installed in the memory 114 from the non-transient storage medium by the non-transient storage medium being mounted in a drive device (not illustrated) of the information processing device 100. The hardware processor is not limited to one configured as a single circuit, and may be configured as one hardware processor that is a combination of a plurality of independent circuits to realize each function. Further, a plurality of components may be integrated into one hardware processor to realize each function.

### Processing Flow of Information Processing Device

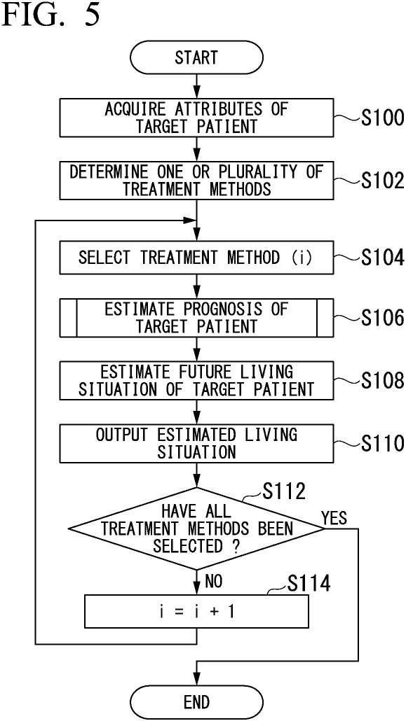

Hereinafter, process of each function by the processing circuitry 120 of the information processing device 100 will be described with reference to the flowchart. FIG. 5 is a flowchart illustrating a flow of a series of process of the processing circuitry 120 according to the first embodiment.

First, the acquisition function 121 acquires attributes of the target patient P1 from the terminal device 10 via the communication interface 111 (step S100).

The attributes of the target patient P1 include a plurality of properties or features, such as a current state of the target patient P1, a transition of a state after the target patient P1 visits a hospital, a transition of a state during follow-up of the target patient P1, a medical history, and hereditary information. Some of the plurality of properties or features may be omitted or replaced with other properties or features that have not been illustrated. For example, the attributes of the target patient P1 may include a treatment method, a treatment history, and the like.

The current state of the target patient P1 includes, for example, age, sex, weight, blood type, vital signs, comorbidities, and expected complications. The transition of the state after the target patient P1 visits the hospital includes, for example, body weight, cardiac function state, respiratory state, metabolic state, image parameters including disease features, and non-image parameters indicating disease features. The transition of the state during the follow-up of the target patient P1 includes, for example, body weight, cardiac function state, respiratory state, metabolic state, image parameters including disease features, and non-image parameters indicating disease features.

For example, it is assumed that the medical worker P2 such as a doctor questions the target patient P1 or his or her family about the current state, the transition of the state, a medical history, the hereditary information, and the like of the target patient P1, and inputs the result of the questioning to the terminal device 10. In this case, the terminal device 10 transmits the input information to the information processing device 100 as an attribute of the target patient P1. When the communication interface 111 receives the attribute of the target patient P1 from the terminal device 10, the acquisition function 121 of the information processing device 100 acquires the attribute from the communication interface 111.

Then, the estimation function 122 determines one or a plurality of treatment methods that can be applied to the target patient P1 (step S102). For example, the estimation function 122 may determine the treatment method on the basis of an input to the terminal device 10 performed by the medical worker P2. Further, the estimation function 122 may determine the treatment method on the basis of the attributes of the target patient P1 (particularly, the current state of the target patient P1).

For example, the output control function 22 of the terminal device 10 causes a plurality of treatment methods that can be applied to the target patient P1 to be displayed on the display 13a. The medical worker P2 (particularly, a doctor) selects one or a plurality of treatment methods from the plurality of treatment methods displayed on the display 13a, and inputs a result of the selection to the input interface 12. The communication control function 23 transmits the selection result of the treatment method input to the input interface 12 by the medical worker P2 to the information processing device 100 via the communication interface 11. In response thereto, the acquisition function 121 of the information processing device 100 acquires the selection result of the treatment method selected by the medical worker P2 from the terminal device 10 via the communication interface 111. The estimation function 122 determines the selection result of the treatment method acquired by the acquisition function 121, that is, the treatment method selected by the medical worker P2 as a treatment method that can be applied to the target patient P1.

Then, the estimation function 122 selects any one treatment method (i) from one or a plurality of determined treatment methods (step S104). i is a temporary internal parameter in the processing circuitry 120, and is a so-called temporary parameter.

Then, the estimation function 122 estimates prognosis of the target patient P1 when the treatment method (i) has been applied (step S106).

### Subprocess

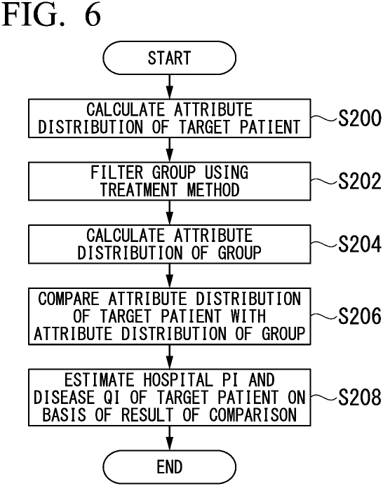

Hereinafter, a process of S106 will be described in detail. FIG. 6 is a flowchart illustrating a flow of a process of estimating the prognosis of the target patient P1. This flowchart corresponds to the process (sub-process) of S106.

First, the estimation function 122 calculates a distribution quantitatively representing attributes (hereinafter referred to as an attribute distribution) of the target patient P1 acquired by the acquisition function 121 (step S200). The attribute distribution of the target patient P1 is an example of a “second distribution”.

For example, the estimation function 122 converts each of a plurality of attributes of the target patient P1 into a quantitative value according to a guideline determined by a government agency or the like or a diagnosis criterion determined by each medical institution, and calculates the quantified attribute as a distribution. The estimation function 122 may quantify the attributes of the target patient P1 using a predetermined database or may quantify the attributes of the target patient P1 using statistics or machine learning (deep learning, or the like).

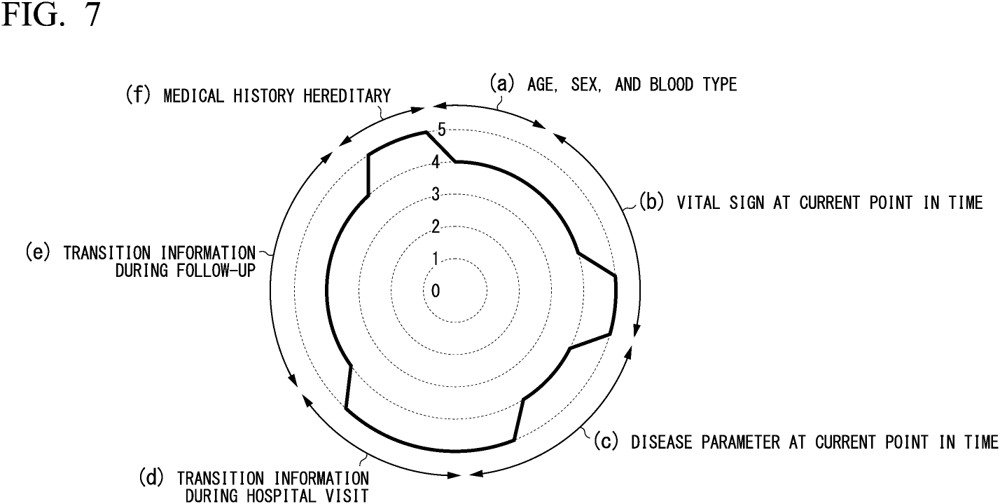

FIG. 7 is a diagram illustrating an example of the attribute distribution. As shown, for example, the attribute distribution may be represented as a radar chart in which the degree of each attribute is graded in six steps from 0 to 5. That is, the value of each attribute may be normalized so that the minimum value becomes 0 and the maximum value becomes 5, and then expressed as a distribution like a radar chart. In area (a) in the attribute distribution, for example, quantitative values of age, sex, and blood type are plotted. In area (b), for example, the quantitative value of a vital sign at a current point in time is plotted. In area (c), for example, the quantitative value of a disease parameter (an image parameters or a non-image parameter) at a current point in time is plotted. In area (d), for example, a quantitative value of transition information during hospital visit is plotted. In area (e), for example, the quantitative value of transition information during follow-up is plotted. In area (f), for example, a quantitative value of the medical history or the hereditary information is plotted.

Attributes of (b) and (c) (for example, the weight, a vital sign, a comorbidity, and a parameter indicating a disease feature) are factors that can be controlled (that is, control factors) before the target patient P1 receives treatment, after the target patient P1 receives treatment, or in a process of the treatment. Attributes (for example, age, blood type, medical history, and family history) of (a), (d), (e), and (f) are factors that cannot be controlled (that is, non-control factors) before and after during treatment of the target patient P1. The control factor is an example of a “first factor”, and the non-control factor is an example of a “second factor”.

In the example of FIG. 7, a case in which the attribute distribution is the radar chart has been described, but the present invention is not limited thereto. For example, the attribute distribution may be represented by other statistical charts such as a histogram, a stacked graph, or a heatmap. Further, the number of steps of the attribute is not limited to 5, and may be 4 or less, or may be 6 or more.

Further, the estimation function 122 may integrate all the attributes of the target patient P1 to obtain one index value (scalar value), instead of or in addition to calculating the attributes of the target patient P1 as a distribution. For example, when the number of attributes of the target patient P1 is n, the estimation function 122 may set a sum T(i) = Σαi × τ(i) in which a degree of influence αi on a disease quality index (QI) in each attribute (τ(i); i=1 ~ n) has been considered, as an index value representing all the attributes of the target patient P1. The disease QI will be described below.

The description is returned to the flowchart of the subprocess of FIG. 6. Then, the estimation function 122 filters patients in a population using the treatment method (i) selected in the process of S104 (step S202). That is, the estimation function 122 extracts, as a sample, a patient to which the treatment method (i) selected in the process of S104 has been applied from the population.

The population consists of the medical worker P2 who is trying to treat the target patient P1 or a plurality of patients treated in the past at a medical institution at which the medical worker P2 works. The medical institution may be, for example, a hospital, a clinic, or other facility in which medical care is provided. Further, the population may also be a medical statistical patient population. The population can be stratified (also referred to as classified, grouped, or clustered) into a plurality of groups on the basis of a hospital performance index (PI) and/or the disease QI.

The hospital PI is an index value regarding a temporal or economic cost spent by each patient in the population, such as the number of hospitalization days or a treatment cost. From another aspect, the hospital PI is an index value regarding a temporal or economic cost spent by the medical institution, such as the number of days of hospital stay or a medical fee. The hospital PI is an example of a “first index value”.

The disease QI is an index value for measuring a treatment effect of how much a disease of each patient in the population has been treated when the patient in the population is treated according to a certain treatment method. For example, when the disease of the patient is cancer, the disease QI may be a 5-year survival rate, the number of days of postoperative hospital stay, a recurrence rate, cancer survival rate, a percentage of breast conserving surgery, and the like. When the disease of the patient is acute myocardial infarction, the disease QI may be an average number of days of hospital stay, and the like. When the disease of the patient is diabetes, the disease QI may be a hemoglobin Alc (HbAlc) improvement rate, the number of patient referrals, the number of reverse patient referrals, and the like. When the disease of the patient is pneumonia, the disease QI may be an average number of days of hospital stay, an initial treatment success rate, and the like. The disease QI is an example of a “second index value”.

For example, when the treatment method (i) selected in the process of S 104 is method “AAA”, the estimation function 122 extracts, as samples, a plurality of patients who have received the treatment method “AAA” in the pas from the population.

Then, the estimation function 122 stratifies a plurality of patients (that is, samples) extracted from the population on the basis of the treatment method (i) into a plurality of groups, and calculates an attribute distribution of each stratified group (step S204).

For example, the estimation function 122 sets one or a plurality of index values as the hospital PIs and/or disease QIs from among a plurality of index values for measuring treatment effects prior to calculation of the attribute distribution of each group of samples.

When the hospital PI and/or the disease QI is selected, the estimation function 122 stratifies the samples to which the treatment method (i) has been applied into a plurality of groups on the basis of the selected hospital PI and/or disease QI. The estimation function 122 may perform such a stratification process at another timing different from that in the present flowchart. The other timing is, for example, a holiday of a medical institution or a night when there are relatively few outpatients. That is, when a process of the present flowchart is started, samples or a population as a sample extraction source may have already been stratified into a plurality of groups.

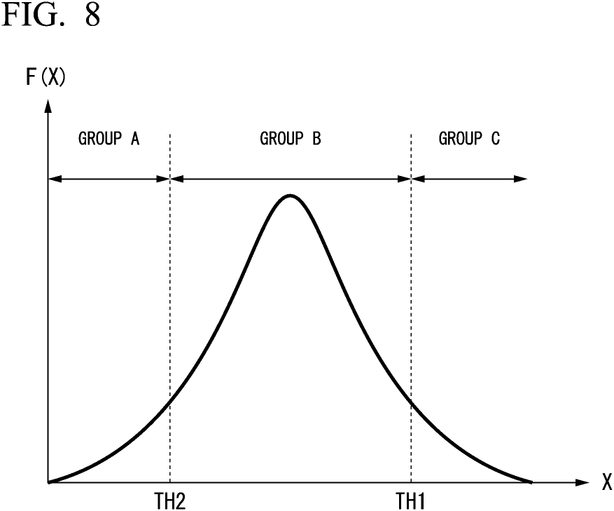

FIG. 8 is a diagram illustrating the stratification process. As illustrated in the figure, for example, the estimation function 122 calculates a probability density distribution F(X) of the sample when the hospital PI or the disease QI is set as a probability variable X. The estimation function 122 stratifies the samples into a plurality of groups according to a certain criterion on the probability density distribution F(X).

For example, the estimation function 122 may classify a group in which the probability variable X is smaller than a second threshold value TH2 into group A, may classify a group in which the probability variable X is equal to or larger than the second threshold value TH2 and smaller than a first threshold value TH1 into group B, and may classify a group in which the probability variable X is equal to or larger than the first threshold value TH1 into group C. The first threshold value TH1 and the second threshold value TH2 may be, for example, fixed values determined on the basis of a medical statistical result or guideline, or may be reference values obtained by adding or subtracting a certain margin to or from a national average or an average within each medical institution. The first threshold value TH1 and the second threshold value TH2 may have standard deviations such as ± 1σ ± 2σ, and ± 3σ. The number of threshold values is not limited to two, and may be one or may be three or more. That is, the number of groups may be two or may be four or more.

For example, when a recurrence rate of cancer is selected as the disease QI, the estimation function 122 extracts a cancer patient or a patient to which a cancer treatment method has been applied as a sample from the population through filtering, and calculates the probability density distribution F(X) with a recurrence rate of the cancer of the extracted sample as the probability variable X. The estimation function 122 stratifies the samples into, for example, a total of three groups A, B, and C on the probability density distribution F(X) regarding the recurrence rate of cancer. In this case, group A is a group with a low recurrence rate of the cancer, group B is a group with a higher recurrence rate of the cancer than group A, and group C is a group with a higher recurrence rate of the cancer than group B.

The estimation function 122 calculates the attribute distribution of each group when the samples are stratified into the plurality of groups. For example, the estimation function 122 averages the attribute distributions of the plurality of respective patients included in each group, and sets an averaged attribute distribution as the attribute distribution of each group. Specifically, when 100 patients are included in group A, the estimation function 122 averages attribute distributions of the 100 patients, and sets one attribute distribution obtained by averaging the attribute distributions of the 100 patients as an attribute distribution of group A. Similarly, the estimation function 122 may averages attribute distributions of a plurality of patients in another group such as group B or group C to calculate an attribute distribution of each group. The attribute distribution of each group is an example of a “first distribution”.

The description is returned to the flowchart of the subprocess of FIG. 6. Then, the estimation function 122 compares the attribute distribution of the target patient P1 with the attribute distribution of each of the plurality of groups stratified from the samples (step S206).

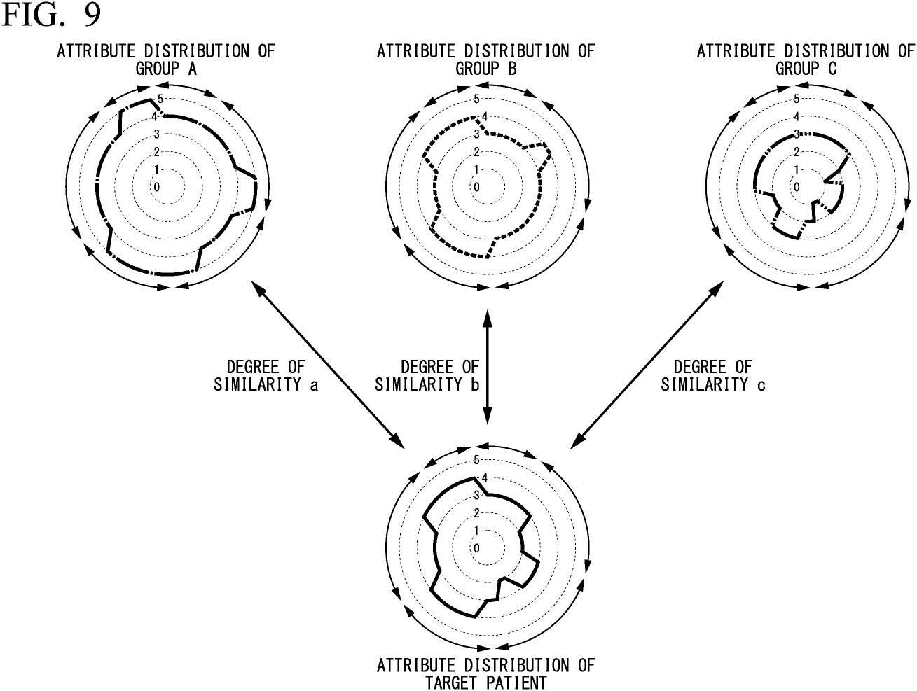

FIG. 9 is a diagram illustrating a method of comparing attribute distributions. For example, it is assumed that there are attribute distributions of three groups including group A, group B, and group C. In this case, the estimation function 122 compares the attribute distribution of the target patient P1 with the attribute distribution of each of group A, group B, and group C, and calculates a degree of similarity between the attribute distributions.

For example, the estimation function 122 calculates, as the degree of similarity a, the degree of similarity between a graphic shape of the attribute distribution of the target patient P1 and a graphic shape of the attribute distribution of group A when the attribute distribution is a chart in which features appear in a shape like a radar chart. Similarly, the estimation function 122 calculates the degree of similarity between the graphic shape of the attribute distribution of the target patient P1 and the graphic shape of the attribute distribution of the group B as a degree of similarity b, and calculates a degree of similarity between the graphic shape of the attribute distribution of the target patient P1 and the graphic shape of the attribute distribution of group C as a degree of similarity c. The degree of similarity increases as the two attribute distributions that are comparison targets are close to similar shapes. Further, when the attribute distribution is a chart in which features appear in colors or shades like a heat map, the estimation function 122 may calculate a distance in colors or shades (so-called a color difference) between the two attribute distributions, which are comparison targets, as the degree of similarity. Specifically, the estimation function 122 may calculate a color histogram of each attribute distribution, and calculate the degree of similarity between the two attribute distributions using the Euclidean distance or the degree of cosine similarity of the color histogram.

The description is returned to the flowchart of the subprocess of FIG. 6. Then, the estimation function 122 estimates the index value of at least one of the hospital PI and the disease QI regarding the target patient P1 or both the index values as the prognosis of the target patient P1 on the basis of a result of a comparison between the attribute distribution of the target patient P1 and the attribute distribution of each group (step S208).

For example, the estimation function 122 estimates the hospital PI used when a group having a highest degree of similarity of the attribute distribution among the plurality of groups compared with the attribute distribution of the target patient P1 is stratified, as the hospital PIregarding the target patient P1, and estimates the disease QI used when the group having the highest degree of similarity of the attribute distribution is stratified, as the disease QI for the target patient P1.

For example, in FIG. 9, it is assumed that the degree of similarity b is the highest. In this case, the estimation function 122 estimates the recurrence rate of the cancer used when group B is stratified from the sample as the disease QI of the target patient P1. Group B is a group in which the recurrence rate of cancer is equal to or higher than a second threshold value TH2 and smaller than a first threshold value TH1. Therefore, a value estimated as the recurrence rate of the cancer of the target patient P1 is in a range from the second threshold value TH2 to the first threshold value TH1.

Further, the estimation function 122 may consider a degree of influence βi on the disease QI in each attribute (τ(i); i = 1 ~ n) when estimating the disease QI of the target patient P1. Specifically, the estimation function 122 may add a product of an arbitrary conversion coefficient γ and a sum Σ{βi × (f(i) -τ(i))} to the estimated disease QI in a certain reference range. The degree of influence βi is a coefficient for weighting the attribute to which more attention is to be paid among the attributes of the target patient P1. Further, the estimation function 122 may estimate the disease QI on the basis of a similarity probability obtained by machine learning or deep learning.

Further, the estimation function 122 may calculate statistical values such as the average, the maximum, and t minimum of all hospital PIs and disease QIs of a plurality of groups compared with the attribute distribution of the target patient P1. For example, when the plurality of groups compared with the attribute distribution of the target patient P1 are three groups A, B, and C, the estimation function 122 may calculate an average of the hospital PIs or an average of the disease QIs of group A, B, and C. Accordingly, the flowchart of the subprocess of FIG. 6 ends.

The description is returned to a main flowchart in FIG. 5. When the estimation function 122 estimates the hospital PI and/or the disease QI as the prognosis of the target patient P1, the estimation function 122 estimates a future living situation (daily life) of the target patient P1 to which the treatment method (i) has been applied, on the basis of the prognosis or attributes of the target patient P1 (step S108).

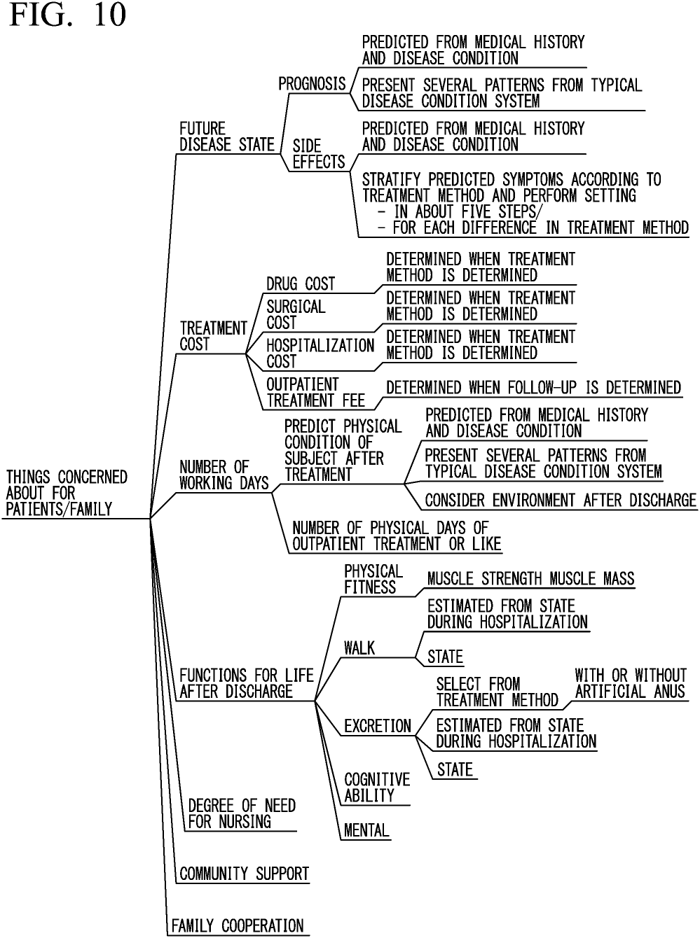

FIG. 10 is a diagram illustrating an example of matters estimated as the future living situation. As illustrated in the figure, the estimation function 122 may estimate the respective matters ((1) future disease state, (2) treatment cost, (3) number of working days, (4) physical or certified function for life after treatment, (5) family cooperation required after treatment, (6) degree of necessity of nursing, and (7) community support) displayed on the above-described intention input screen, as the future living situation of the target patient P1. These matters are estimated by using a database, statistics, and machine learning (including deep learning).

For example, the estimation function 122 may set the survival rate, recurrence rate, or the like estimated as the disease QI as a future disease condition of the target patient P1, and estimate (1) future disease state (prognosis or side effects) on the basis of the future disease condition of the target patient P1 and current attributes (a medical history, and the like) of the target patient P1.

Further, the estimation function 122 may estimate a drug cost, a surgery cost, a hospitalization cost, an outpatient treatment cost, and the like according to the treatment method (i) as (2) treatment cost or estimate various expenses required for follow-up according to the number of follow-ups. With regard to drugs, variations in some drug costs may be estimated depending on whether generic drugs are used, and used jigs (for example, a type of stoma or a type of implant).

Further, the estimation function 122 may estimate the number of days during which the target patient P1 is in a good physical condition after treatment, the number of days required for outpatient treatment, and the like, on the basis of the disease QI of the target patient P1 such as the survival rate or the recurrence rate and the attributes of the target patient P1 such as a medical history, as (3) number of working days. Further, the estimation function 122 may estimate the number of working days further in consideration of a rehabilitation environment or lift information after discharge. The number of days required for outpatient treatment may be estimated from the number of follow-ups, the number of treatment days, and the like according to the treatment method (i).

Further, the estimation function 122 may estimate physical fitness, walking, excretion, cognitive ability, mentality, and the like as (4) physical or certified function for life after treatment. The physical fitness may be estimated from a muscle mass, a physical condition during hospitalization, and a rehabilitation state. The walking (are you bedridden?) is estimated according to a life level of nursing certification by a database, statistics, and machine learning (including deep learning) according to a state of the physical fitness. The excretion is estimated according to a life level of nursing certification from a treatment method including whether or not an artificial anus can be installed, and a physical condition of a person (the target patient P1). The excretion may be estimated further in consideration of management willingness of the person, in addition to the physical condition of the person.

Further, the estimation function 122 may comprehensively consider the estimation results (1) to (4) to estimate (5) family cooperation required after treatment, (6) degree of necessity of nursing care, and (7) community support.

The description is returned to the main flowchart in FIG. 5. Then, the output control function 124 outputs the future living situation of the target patient P1 estimated by the estimation function 122 (hereinafter referred to as an estimated living situation) via the output interface 113 (step S110). The communication control function 125 may transmit the estimated living situation to the terminal device 10 via the communication interface 111, instead of or in addition to the output control function 124 outputting the estimated living situation. In this case, the estimated living situation is displayed on the display 13a of the terminal device 10.

Next, the estimation function 122 determines whether or not all the treatment methods that can be applied to the target patient P1 have been selected (step S112). When all the treatment methods have been selected, the estimation function 122 ends the process of the present flowchart.

On the other hand, when all the treatment methods have not been selected yet, the estimation function 122 increments a temporary parameter i (step S114) and returns the process to S104. That is, the estimation function 122 reselects a treatment method that has never been selected as a new treatment method (i). Accordingly, the estimated living situation of the target patient P1 is output for each treatment method. As a result, the target patient P1 or his or her family can compare an intention input to the terminal device 10 with the estimated living situation, and can select a treatment method suitable for the intention of the target patient P1 or his or her family.

According to the first embodiment described above, the information processing device 100 calculates the attribute distribution of the target patient P1, extracts, as samples, patients to which the same treatment method (i) as that for the target patient P1 has been applied from a certain population, stratifies the samples into a plurality of groups, and calculates the attribute distribution of each stratified group. The information processing device 100 compares the attribute distribution of each group (an example of a “first distribution”) with the attribute distribution of the target patient P1 (an example of a “second distribution”), and estimates at least one or both of the index values of the hospital PI (an example of a “first index value”) and the disease QI (an example of the “second index value”) regarding the target patient P1, as the prognosis of the target patient P1, on the basis of a result of the comparison. The information processing device 100 estimates the future living situation of the target patient P1 for each treatment method on the basis of at least the prognosis of the target patient P1. The information processing device 100 outputs the estimated living situation via its own output interface 113 or transmits the estimated living situation to the terminal device 10. This makes it possible for the target patient P1 or his or her family to compare the intention input to the terminal device 10 with the future living situation estimated by the information processing device 100 (the estimated living situation) and easily imagine a daily life after the treatment. As a result, the target patient P1 or his or her family can select a treatment method for desired daily life. In other words, because future life after treatment can be understood, the treatment can be received with peace of mind not only for the patient but also for the family.

### Second Embodiment

Hereinafter, a second embodiment will be described. The second embodiment differs from the first embodiment in that an information gap between the future living situation desired by the target patient P1 or his or her family and the future living situation estimated by the information processing device 100 (the estimated living situation) is estimated, and different information is output according to the information gap. Hereinafter, differences from the first embodiment will be mainly described, and description of points common to the first embodiment will be omitted. In the description of the second embodiment, the same parts as those of the first embodiment will be described with the same reference numerals.

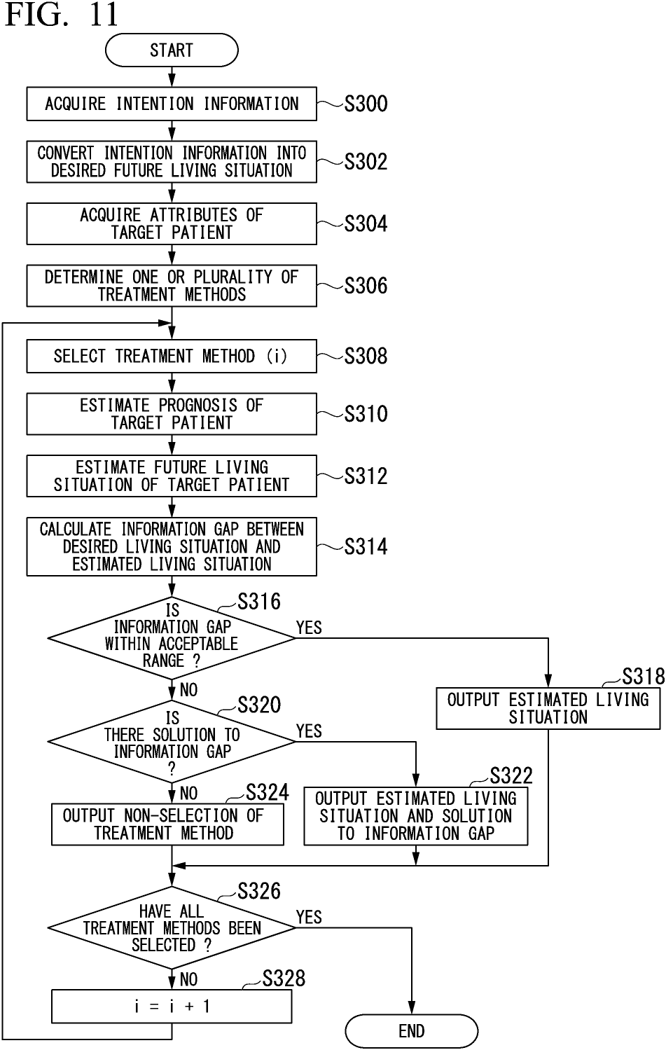

FIG. 11 is a flowchart illustrating a flow of a series of process of the processing circuitry 120 according to the second embodiment.

First, the acquisition function 121 acquires intention information of the target patient P1 or his or her family from the terminal device 10 via the communication interface 111 (step S300). For example, the acquisition function 121 may acquire information input to the intention input screen of FIG. 3 as the intention information.

When the intention of the target patient P1 or his or her family is explored, a decision-making process note or tool (selection of artificial hydration and nutrition (AHN), selection of artificial dialysis, and conditions for attachment and detachment of a ventilator) as advocated by the clinical ethics network Japan, advance care planning tools, or the like may be used. The economic and social environment surrounding the target patient P1 or his or her family may be acquired as background data of the intention information.

Further, it is assumed that the medical worker P2 hears the intention regarding treatment from the target patient P1 or his or her family, and inputs a result of the questioning to the terminal device 10. In this case, the acquisition function 121 may acquire the hearing result input to the terminal device 10 by the medical worker P2 as the intention information of the target patient P1 or his or her family.

Then, the estimation function 122 converts the intention information of the target patient P1 or his or her family into the future living situation desired by the target patient P1 or his or her family (hereinafter referred to as desired living situation) (step S302). For example, the estimation function 122 converts qualitative information such as the intention information into quantitative information as the desired living situation.

For example, on the intention input screen, the options such as “of great concern”, “of concern”, and “of less concerned” are provided, and when these options are answered, the estimation function 122 may score the matters of (1) to (7) according to the answered options.

Further, for example, when the hearing result is acquired as the intention information, the estimation function 122 may convert a sentence representing the hearing result into a relative or absolute score on the basis of a keyword by using a natural language process. This score conversion may be performed by using a database (a dictionary) corresponding to the keyword, or may be performed using machine learning represented by deep learning or the like.

The respective questions in the intention input screen may be further subdivided as illustrated in FIG. 10. For example, questions regarding (1) future disease state may be further provided with items of prognosis or side effects. The questions regarding (2) treatment cost may be further provided with items such as a drug cost, a surgery cost, a hospitalization cost, an outpatient treatment cost. The questions regarding (3) number of working days may be further provided with items such as the number of days during which the target patient P1 is in a good physical condition after treatment, and the number of days required for outpatient treatment. Questions regarding (4) physical or certified function for life after treatment may be further provided with items such as physical fitness, walking, excretion, cognitive ability, and mentality. The same applies to the questions regarding (5) family cooperation required after treatment, questions regarding (6) degree of necessity of nursing care, and questions regarding (7) community support.

When the respective questions are subdivided in the intention input screen and a specific numerical value such as an acceptable cost or a desired number of days can be input, the estimation function 122 sets the specific numerical value input to the intention input screen as a score.

Thus, the estimation function 122 uses the quantitative information converted from the qualitative information such as the intention information, as the desired living situation. The desired living situation is treated as a comparison target of the estimated living situation in a process to be described below. The desired living situation is an example of a “first living situation”, and the estimated living situation is an example of a “second living situation”.

FIG. 12 is a diagram illustrating an example of the desired living situation. As illustrated in the figure, the desired living situation may be information or data obtained by scoring intentions for the respective items (1) to (7) or items obtained by subdividing these. The illustrated example shows that the target patient P1 or his or her family considers a treatment in which a score regarding (2) treatment cost is level 2 as an ideal (desired) one, an acceptable lower limit of the score is set to level 2, and an acceptable upper limit is set to level 5. The upper limit value and the lower limit value may be set by the target patient P1 or his or her family. This means that a treatment method in which the score of each item is within a range of the upper and lower limit values is desirable for the target patient P1 or his or her family.

The description is returned to the flowchart in FIG. 11. Then, the acquisition function 121 acquires the attributes of the target patient P1 from the terminal device 10 via the communication interface 111 (step S304).

Then, the estimation function 122 determines one or a plurality of treatment methods that can be applied to the target patient P1 (step S306).

Then, the estimation function 122 selects any one treatment method (i) from the one or a plurality of determined treatment methods (step S308).

Then, the estimation function 122 estimates prognosis of the target patient P1 when the treatment method (i) has been applied (step S310). Since a method of estimating the prognosis is the same as the flowchart of the subprocess of FIG. 6 described above, description thereof will be omitted here.

Then, the estimation function 122 estimates the future living situation of the target patient P1 to which the treatment method (i) has been applied, on the basis of the prognosis or attributes of the target patient P1 (step S312).

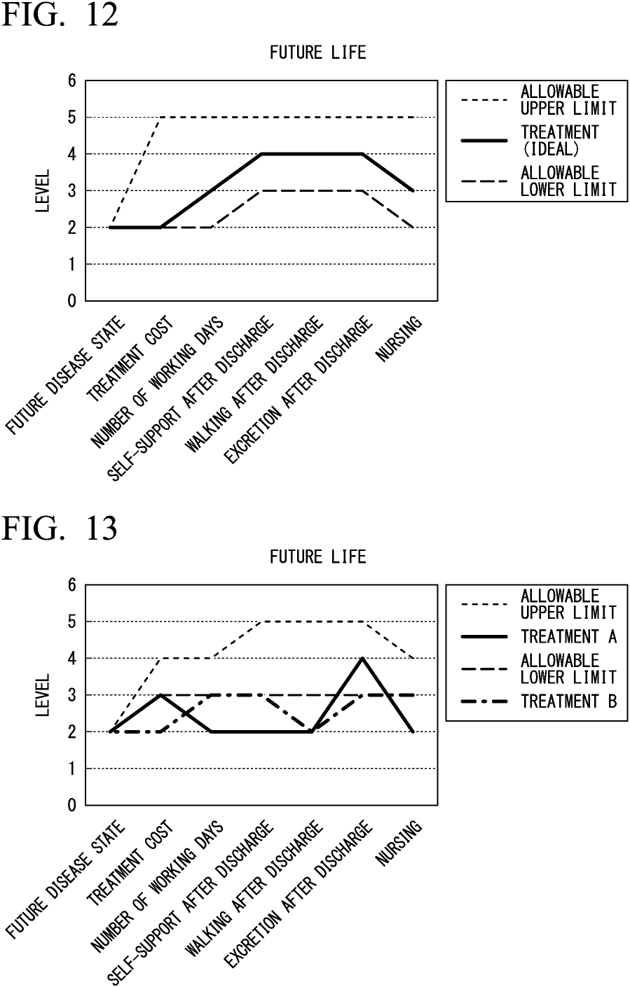

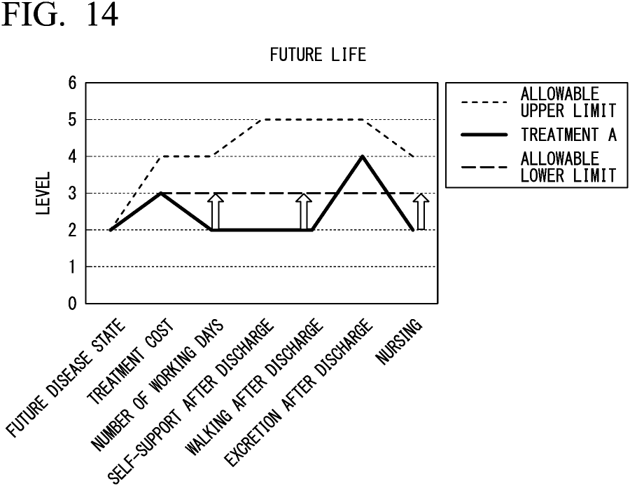

FIG. 13 is a diagram illustrating an example of the estimated living situation. The estimated living situation may be information or data obtained by scoring the respective items (1) to (7) or items obtained by subdividing these, like the desired living situation. In the illustrated example, it is shown that, when a certain treatment method A has been applied to the target patient P1, the score regarding (2) treatment cost becomes level 3, and when a certain treatment method B has been applied to the target patient P1, the score regarding (2) treatment cost becomes level 2. In FIG. 13, the lower limit of the score is set to level 2 for (1) future disease state and to level 3 for all other items including (2) treatment cost.

The description is returned to the flowchart in FIG. 11. Then, the determination function 123 calculates an information gap between the desired living situation converted from the intention information in a process of S302 and the future living situation of the target patient P1 estimated in the process of S312 (that is, the estimated living situation) (step S314).

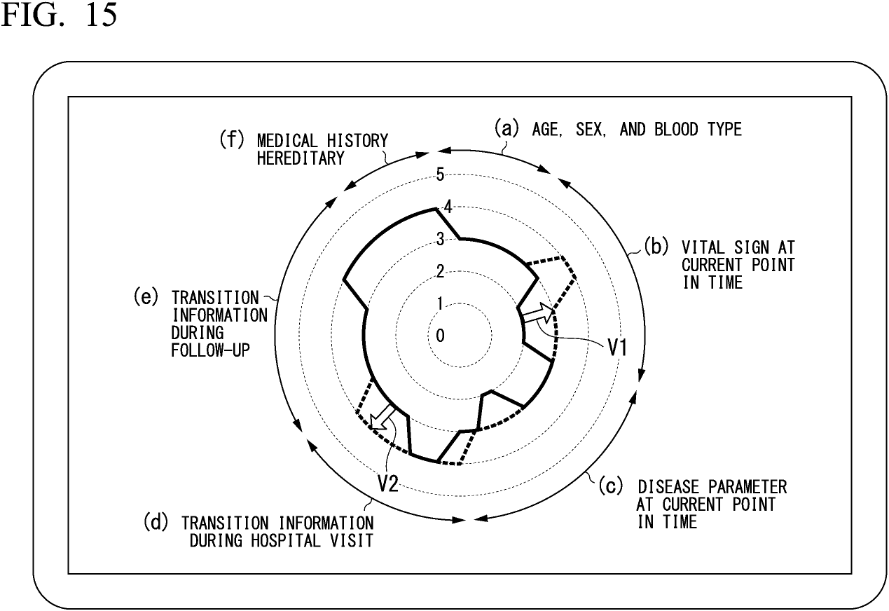

FIG. 14 is a diagram illustrating an example in which the information gap has been generated. In the example of FIG. 14, the estimated living situation when the treatment method A of FIG. 13 has been applied to the target patient P1 is compared with the lower limit of the score digitized as the desired living situation. For example, for (3) number of working days, the lower limit of the score is set to level 3, but is set to level 2 when the treatment method A has been applied to the target patient P1. Therefore, the determination function 123 calculates an information gap for one level regarding a score regarding (3) number of working days. Similarly, the determination function 123 calculates an information gap for scores regarding other matters.

The description is returned to the flowchart in FIG. 11. Then, the determination function 123 determines whether or not the calculated information gap is within an acceptable range (step S316). The acceptable range is a range of upper and lower limits set by the target patient P1 or his or her family. In the example of FIG. 14, when the treatment method A is applied to the target patient P1, at least the score regarding (3) number of working days is smaller than the lower limit. Therefore, the determination function 123 determines that the information gap is out of the acceptable range.

When the information gap is within the acceptable range, the output control function 124 outputs the future living situation of the target patient P1 estimated by the estimation function 122, that is, the estimated living situation via the output interface 113 (step S318). The communication control function 125 may transmit the estimated living situation to the terminal device 10 via the communication interface 111, instead of or in addition to the output control function 124 outputting the estimated living situation. In this case, the estimated living situation is displayed on the display 13a of the terminal device 10.

On the other hand, when the information gap is out of the acceptable range, the determination function 123 further determines whether or not there is a solution to the information gap (step S320).

As described above, there are control factors and non-control factors in the attributes of the target patient P1, and it is possible to reduce the information gap even with the same treatment method by adjusting the control factors in some cases. For example, when the target patient P1 has lost weight, the information gap may be narrower than when the target patient P1 has not lost weight even with the same treatment method, or when the target patient P1 stops smoking, the information gap may be narrower than when the target patient P1 continues to smoke even with the same treatment method. Thus, even the same treatment method can be suitable for the desired living situation depending on efforts of the target patient P1.

Therefore, when the determination function 123 determines that the information gap is out of the acceptable range, the estimation function 122 virtually adjusts (changes) control factors such as a body weight included in the attributes of the target patient P1 acquired in a process of S304. The estimation function 122 estimates the prognosis of the target patient P1 on the basis of the attributes of the target patient P1 in which the control factor has been adjusted. That is, the estimation function 122 simulates the prognosis of the target patient P1 on the basis of the attributes of the target patient P1 in which adjustment to a control factor different from the actual one has been performed. The estimation function 122 estimates the future living situation of the target patient P1 to which the treatment method (i) has been applied, on the basis of the simulated prognosis of the target patient P1 or the attributes of the target patient P1. The future living situation of the simulated target patient P1 is an example of a “third living situation”.

The determination function 123 calculates the information gap between the desired living situation converted from the intention information in the process of S302 and the future living situation of the simulated target patient P1, and determines whether or not the information gap is within the acceptable range. When the information gap is within the acceptable range, the determination function 123 determines that changing the control factor is a solution to the information gap under a condition before the simulation.

When the information gap is out of the acceptable range, but there is a solution to the information gap, the output control function 124 outputs the solution to the information gap together with the estimated living situation via the output interface 113 (step S322).

FIG. 15 is a diagram illustrating an output example of the solution to the information gap. For example, as indicated by arrows V1 and V2, it is assumed that the information gap is within the acceptable range when the attributes of (b) and the attributes of (d) are adjusted through simulation in the attribute distribution of the target patient P1. Both of these attributes are control factors. Therefore, the output control function 124 may output information for encouraging improvement of attributes such as vital signs or transition information during hospital visit via the output interface 113 without changing the treatment method. For example, when the weight of the target patient P1 is reduced and the information gap is narrowed, the output control function 124 may output information for encouraging the target patient P1 to reduce the weight. Further, when the target patient P1 suffers from a complication, the complication is reduced, and the information gap is narrowed, the output control function 124 may output information for encouraging the target patient P1 to reduce the complication. Further, the communication control function 125 may transmit the solution to the information gap to the terminal device 10 via the communication interface 111. In this case, the solution to the information gap is displayed on the display 13a of the terminal device 10.

When the information gap is out of the acceptable range and there is no solution to the information gap, the output control function 124 outputs the fact that the treatment method (i) selected in a process of S308 cannot be selected (hereinafter referred to as unadoption notification) via the output interface 113 (step S324). The communication control function 125 may transmit the unadoption notification to the terminal device 10 via the communication interface 111. In this case, the notification of the unadoption of the treatment method (i) is displayed on the display 13a of the terminal device 10.

Then, the estimation function 122 determines whether or not all the treatment methods that can be applied to the target patient P1 have been selected (step S326). When all the treatment methods have been selected, the estimation function 122 ends the process of the present flowchart.

On the other hand, when all the treatment methods have not been selected yet, the estimation function 122 increments the temporary parameter i (step S328) and returns the process to S308.

According to the second embodiment described above, the information processing device 100 determines the information gap between the desired living situation and the estimated living situation, and outputs different information according to the information gap. For example, when the information gap is within the acceptable range or when there is a solution to the information gap even when the information gap is out of the acceptable range, the medical worker P2 including a doctor can explain a result output by the information processing device 100 to the patient P1 or his or her family, to smoothly agree on the treatment method with the target patient P1 or his or her family. On the other hand, when the information gap is out of the acceptable range and there is no solution to the information gap, the medical worker P2 can explain that there is no treatment method on which the target patient P1 or his or her family can agree (cannot agree on or there is not desired treatment method). This makes it possible for the target patient P1 or his or her family to reexamine an intention regarding the treatment method. When the intention is reexamined, the process of the flowchart described above is repeated on the basis of the intention, thereby making it possible to reach the agreement on the target patient P1 or his or her family.

### Other Embodiments

Hereinafter, other embodiments will be described. In the above-described embodiment, a case in which the terminal device 10 and the information processing device 100 are different devices from each other have been described, but the present invention is not limited thereto. For example, the terminal device 10 and the information processing device 100 may be one integrated device. For example, the processing circuitry 20 of the terminal device 10 may include an estimation function 122, an estimation function 122, and an estimation function 122 included in the processing circuitry 120 of the information processing device 100, in addition to the acquisition function 21, the output control function 22, and the communication control function 23. In this case, the terminal device 10 can perform the processes of the various flowcharts described above on a stand-alone basis (offline).

Further, the process of the flowchart of FIG. 11 is described as being executed only by the information processing device 100, but the present invention is not limited thereto. For example, a part of the process of the flowchart of FIG. 11 may be executed by the terminal device 10. Specifically, processes of S316 to S324 may be executed by the terminal device 10, and other processes of S300 to S314, S326, and S328 may be executed by the information processing device 100. In this case, the terminal device 10 may be used by a care manager or an attending physician of the target patient P1, and the information processing device 100 may be used by a clinical department to which a doctor in charge of the care manager, or the attending physician belongs.

Although some embodiments have been described, these embodiments are presented as examples and are not intended to limit the scope of the invention. These embodiments can be implemented in various other forms, and various omissions, replacements, and changes can be made without departing from the gist of the invention. These embodiments or modifications thereof are included in the scope or gist of the invention, as well as in a scope of the invention described in the claims and an equivalent scope thereof.

What is claimed is:

1. An information processing device comprising a processing circuitry configured to:
estimate a future living situation of a target patient for each treatment method to be applied to the target patient on the basis of attributes of the target patient, and
output information based on the living situation via an output unit.

2. The information processing device according to claim 1,
wherein the processing circuitry
estimates a prognosis of the target patient for each treatment method, and
estimates the future living situation of the target patient for each treatment method on the basis of the prognosis.

3. The information processing device according to claim 2,
wherein the processing circuitry
calculates a first distribution quantitatively representing attributes of each of a plurality of groups stratified from a plurality of patients on the basis of diagnostic information of the plurality of patients to which the same treatment method as that for the target patient has been applied,
calculates a second distribution quantitatively representing the attributes of the target patient, and
estimates the prognosis of the target patient on the basis of a comparison between the first distribution and the second distribution.

4. The information processing device according to claim 3, wherein the processing circuitry stratifies the plurality of patients into the plurality of groups on the basis of a first index value regarding a temporal or economic cost of each of the plurality of patients and a second index value regarding treatment effects of each treatment method applied to each of the plurality of patients.

5. The information processing device according to claim 4, wherein the processing circuitry determines at least one of the first index value and the second index value regarding the target patient as the prognosis of the target patient on the basis of on the comparison between the first distribution and the second distribution.

6. The information processing device according to claim 5, wherein the processing circuitry calculates a degree of similarity between the second distribution and the first distribution of each of the plurality of groups, and estimates the first index value of the first distribution having the highest degree of similarity to the second distribution as the first index value regarding the target patient, or estimates the second index value of the first distribution having the highest degree of similarity to the second distribution as the second index value regarding the target patient.

7. The information processing device according to claim 1,
wherein the processing circuitry further
determines whether or not a first information gap has been generated between a first living situation, the first living situation being a future living situation of the target patient desired by the target patient or a family of the target patient, and a second living situation, the second living situation being a future living situation of the target patient estimated by the estimation unit, and
makes information to be output to the output unit different between a case in which the first information gap has been generated or the first information gap has not been generated.

8. The information processing device according to claim 7,
wherein the processing circuitry
further determines whether or not there is a solution to the first information gap when the first information gap has been generated, and
outputs the solution to the first information gap via the output unit when there is a solution to the first information gap.

9. The information processing device according to claim 8,
wherein the attributes of the target patient include a first factor that is able to be controlled by the target patient and a second factor that is unable to be controlled by the target patient, and
the processing circuitry
further estimates a future living situation of the target patient on the basis of the attributes of the target patient whose first factor has been adjusted when the first information gap has been generated,
determines whether or not a second information gap has been generated between the first living situation and a third living situation, the third living situation being a future living situation of the target patient estimated when the first factor has been adjusted,
determines that there is a solution to the first information gap when the second information gap has not been generated, and
outputs there being an adjustment of the first factor in the solution to the first information gap via the output unit.

10. A medical information processing method using a computer, the medical information processing method comprising:
estimating a future living situation of a target patient for each treatment method to be applied to the target patient on the basis of attributes of the target patient, and
outputting information based on the living situation via an output unit.
